(19) United States
(12) Patent Application Publication
(10) Pub. No.: US 2023/0270131 A1
(43) Pub. Date: Aug. 31, 2023

(54) Functional Candy with Special Effect on Gastrointestinal Regulation and Industrialized Production Method Thereof

(71) Applicant: Shenzhen Pandalu Biotechnology Co., Ltd., Shenzhen (CN)

(72) Inventors: Jianxian ZHENG, Shenzhen (CN); Hao WANG, Shenzhen (CN); Yuting WANG, Shenzhen (CN)

(21) Appl. No.: 17/929,384

(22) Filed: Sep. 2, 2022

(30) Foreign Application Priority Data
Dec. 30, 2021 (CN) 202111662396.2

(51) Int. Cl. A23G 3/42; A23G 3/44; A23G 3/48; A23G 3/34; B01D 61/14; B01D 69/02; A61K 36/28; A61K 36/07; A61K 9/00; A01G 22/25; A01C 21/00

(52) U.S. Cl. CPC A23G 3/42; A23G 3/44; A23G 3/48; A23G 3/0027; B01D 61/145; B01D 69/02; A61K 36/28; A61K 36/07; A61K 9/0056; A01G 22/25; A01C 21/00; B01D 2325/20

(57) ABSTRACT

The present disclosure provides a functional candy with a special effect on gastrointestinal regulation and an industrialized production method thereof. The method includes: step 1, preparing a yacon polysaccharide-peptide-zinc chelate with a high activity; step 2, mixing 1 part to 5 parts of the yacon polysaccharide-peptide-zinc chelate with 10 parts to 20 parts of a chicory extract powder, 10 parts to 20 parts of a Hericium erinaceus extract powder, 5 parts to 15 parts of xylooligosaccharide, 5 parts to 15 parts of fructooligosaccharide, and 10 parts to 20 parts of water well to prepare a main agent; and step 3, adding an auxiliary agent including other ingredients to the main agent to prepare the functional candy. The present disclosure further provides a functional candy with a special effect on gastrointestinal regulation prepared by the method.

### CROSS REFERENCE TO RELATED APPLICATION

This patent application claims the benefit and priority of Chinese Patent Application No. 202111662396.2, filed on Dec. 30, 2021, the disclosure of which is incorporated by reference herein in its entirety as part of the present application.

### TECHNICAL FIELD

The present disclosure relates to a highly-active yacon polysaccharide-peptide-zinc chelate-based candy in the field of functional active ingredient-containing food and an industrialized preparation method thereof. Specifically, the present disclosure relates to a functional candy with a special effect on gastrointestinal regulation and an industrialized production method thereof.

### BACKGROUND ART

Zinc is one of the essential elements for human body. There are more than 200 types of enzymes in the human body that contain zinc, which are closely-related to the body metabolic activities. Common zinc-containing enzymes include glutamate dehydrogenase, lactate dehydrogenase, and pyruvate oxidase. Zinc plays an extremely important role in human immune response; studies have shown that there is an impaired immune T cell function in the body under zinc deficiency, resulting in mediated immune changes to reduce resistance, thereby making the body susceptible to bacterial infection. In addition, zinc is indispensable for the perception of taste. Zinc deficiency may lead to loss of appetite and dull taste, affecting a nutritional balance of the human body. Therefore, maintaining a zinc balance in the body plays an important role in human health.

Constipation is a relatively-common digestive system disease. Since the feces stays in the intestine for too long time, the intestinal wall absorbs most of water in the feces, resulting in dry and hard feces that is difficult to be excreted. Nowadays, with the rapid development of society, there are a lack of exercise, high-intensity work, unhealthy diet and other bad habits, causing an expanded scope and number of people suffered from the constipation. Constipation is more common in females than males, and in older people than in young adults; the constipation has high morbidity and complex etiology, which generally brings a lot of distress to patients, thus seriously affecting a living quality of the patients. At present, most of constipation patients are treated by drug therapy. Although having a quick effect, the drug therapy has a high recurrence rate and a certain impact on the stability of intestinal flora.

### SUMMARY

An objective of the present disclosure is to provide a highly-active yacon polysaccharide-peptide-zinc chelate-based candy and an industrialized preparation method thereof. Aiming at problems existing in the prior art, the present disclosure provides a functional candy with a special effect on gastrointestinal regulation. The functional candy can effectively solve the constipation, and also has green and natural raw materials, no side effects, and suitability for long-term use.

To achieve the above objective, the present disclosure provides an industrialized production method of a functional candy with a special effect on gastrointestinal regulation. The method includes the following steps: step 1, preparing a yacon polysaccharide-peptide-zinc chelate with a high activity; step 2, mixing the yacon polysaccharide-peptide-zinc chelate obtained in step 1 with a chicory extract powder, a Hericium erinaceus extract powder, xylooligosaccharide, fructooligosaccharide, and water to obtain a main agent; and step 3, adding an auxiliary agent including other ingredients to the main agent to prepare the functional candy.

Preferably, step 1 may specifically include the following steps: step 1.1, cultivating the yacon, applying a zinc-rich nutrient solution to each yacon seedling during a growth process of the yacon seedlings until one week before the yacon matures; step 1.2, preparing a homogenate from a mature yacon fruit as a raw material, weighing, adding water, heating and stirring under heat preservation, adding an enzyme to conduct a reaction, filtering an obtained enzymatic hydrolysate with a centrifuge to remove residues, and conducting inactivation on the enzymatic hydrolysate; step 1.3, adding bioethanol to an inactivated enzymatic hydrolysate, allowing to stand, and filtering to obtain a yacon polysaccharide-peptide-zinc chelate crude extract; and step 1.4, redissolving the yacon polysaccharide-peptide-zinc chelate crude extract in water, and conducting an ultrafiltration membrane treatment to obtain the yacon polysaccharide-peptide-zinc chelate with a high activity.

Preferably, in step 1.1, during cultivating the yacon, when the yacon seedlings grow to 10 cm to 15 cm, 50 mL to 200 mL of the zinc-rich nutrient solution may be applied to each yacon seedling every 3 d to 5 d until one week before the yacon matures; the zinc-rich nutrient solution may include the following components in parts by weight: 1 part to 5 parts of a zinc fertilizer, 1 part to 5 parts of an amino acid, 20 parts to 50 parts of a compound fertilizer, 10 parts to 30 parts of sawdust, 20 parts to 30 parts of wheat bran, and 1000 parts of water; a zinc element form of the zinc fertilizer in the zinc-rich nutrient solution may include any one or more selected from the group consisting of zinc sulfate heptahydrate, a zinc-amino acid chelate, a zinc-rich yeast, and a zinc-EDTA chelate; and the zinc-rich nutrient solution may be applied to the yacon seedlings by foliage spray or root fertilization.

Preferably, in step 1.2, the homogenate may be prepared from the mature yacon fruit as the raw material, weighed, water may be added according to a mass-to-volume ratio of the raw material and water at 1:10 to 1:30, heated to 90° C. to 95° C. and stirred under heat preservation for 2 h to 3 h, the enzyme may be added according to 0.1% to 1% of a mass of the raw material to conduct a reaction at 40° C. to 65° C. and a pH value of 4.5 to 7.0 for 80 min to 100 min, the obtained enzymatic hydrolysate may be filtered with a centrifuge at 2,000 r/min to 4,000 r/min to remove the residues, and inactivation may be conducted on the enzymatic hydrolysate at 90° C. to 100° C.; and the enzyme may be any one or an enzyme complex of more selected from the group consisting of cellulase, hemicellulase, and pectinase.

Preferably, in step 1.3, 65% to 90% of the bioethanol by mass concentration may be added to the inactivated enzymatic hydrolysate according to a volume ratio of ethanol and the enzymatic hydrolysate at 2:1 to 4:1, allowed to stand, and filtered to obtain the yacon polysaccharide-peptide-zinc chelate crude extract.

Preferably, in step 1.4, the yacon polysaccharide-peptide-zinc chelate crude extract may be redissolved in water according to a mass ratio of the yacon polysaccharide-peptide-zinc chelate crude extract and water at 1:10 to 1:30, and the ultrafiltration membrane treatment may be conducted at 0.22 MPa to 0.30 MPa and 25° C. to 30° C. in a material-liquid volume flow rate of 0.08 L/min to 0.12 L/min using ultrafiltration membranes with molecular weight cut-offs of 40,000 and 30,000 separately to obtain the yacon polysaccharide-peptide-zinc chelate with a high activity and having a molecular weight of 30,000 to 40,000.

Preferably, in step 2, in parts by weight, 1 part to 5 parts of the yacon polysaccharide-peptide-zinc chelate may be mixed with 10 parts to 20 parts of the chicory extract powder, 10 parts to 20 parts of the Hericium erinaceus extract powder, 5 parts to 15 parts of the xylooligosaccharide, 5 parts to 15 parts of the fructooligosaccharide, and 10 parts to 20 parts of the water, and stirred at 50° C. to 60° C. for 20 min to 30 min to obtain the main agent.

Preferably, in step 3, a type of the candy may include any one or more selected from the group consisting of a gelatinous candy, a hard candy, and a tablet candy.

Preferably, when the candy is the gelatinous candy: a gellant may be stirred with water, and cooled to obtain a gelatinous candy auxiliary agent, the main agent and the auxiliary agent may be mixed, stirred evenly, and an obtained material liquid may be poured into a silica gel mold for molding, demolding may be conducted after cooling, and the gelatinous candy may be sealed and packaged under vacuum conditions; and the gellant may be any one or more selected from the group consisting of carrageenan, gelatin, agar, konjac gum, pectin, and gum arabic; when the candy is the hard candy: an auxiliary agent including a sugar or a sugar alcohol and the main agent may be mixed uniformly by heating, and boiled at a high temperature to obtain a material liquid, and the material liquid may be poured into the silica gel mold for molding, dried, and demolded to obtain the hard candy; and the sugar or the sugar alcohol may be any one or more selected from the group consisting of white granulated sugar, fructose, and xylitol; when the candy is the tablet candy: the main agent and the auxiliary agent including the sugar or the sugar alcohol may be mixed, granulated, dried, and sieved, added with a 1% lubricant by mass percentage, mixed evenly and tableted in a tablet press to obtain the tablet candy; and the sugar or the sugar alcohol may be any one or more selected from the group consisting of glucose, isomaltooligosaccharide, maltitol, and erythritol.

The present disclosure further provides a functional candy with a special effect on gastrointestinal regulation produced by the method, including a functional main agent prepared by the following components in parts by weight: 1 part to 5 parts of the yacon polysaccharide-peptide-zinc chelate, 10 parts to 20 parts of the chicory extract powder, 10 parts to 20 parts of the Hericium erinaceus extract powder, 5 parts to 15 parts of the xylooligosaccharide, 5 parts to 15 parts of the fructooligosaccharide, and 10 parts to 20 parts of water; where the yacon polysaccharide-peptide-zinc chelate is a compound with a molecular weight of 30,000 to 40,000, and has 68.9% to 73.8% of a polysaccharide, 25.7% to 26.1% of a peptide, and 150 mg/100 g to 200 mg/100 g of zinc by mass percentage.

The functional candy with a special effect on gastrointestinal regulation and the industrialized production method thereof provided by the present disclosure have the following advantages:

The present disclosure provides a preparation method of a yacon polysaccharide-peptide-zinc chelate with a high activity, and further provides a preparation method of a functional candy with a special effect on gastrointestinal regulation by the chelate and various plant active materials, and an industrialized production method thereof.

In the present disclosure, the yacon polysaccharide-peptide-zinc chelate with a high activity has an extremely high biological value, and is rich in fructose polymer-based prebiotics, peptide compounds, and biological zinc elements. The chelate is a natural active substance that is easily absorbed in the human body and has a significant biological activity. The extracted yacon polysaccharide-polypeptide-zinc chelate is scientifically compatible with various natural plant active ingredients to prepare the functional candy with a special effect on gastrointestinal regulation.

In the present disclosure, the functional candy includes the natural and highly-active yacon polysaccharide-peptide-zinc chelate, as well as various plant extracts that are beneficial for regulating the human gastrointestinal tract; the raw materials are green and natural, without added pigments and additives. In terms of diet, the functional candy can effectively regulate the health of body gastrointestinal tract, and has a significant effect on prevention and treatment of chronic gastrointestinal diseases, especially for people with constipation. In addition, the functional candy with a special effect on gastrointestinal regulation has a desirable taste, meets the public's demand for healthy and delicious food, and is convenient to carry, which has a large market potential.

### DETAILED DESCRIPTION OF THE EMBODIMENTS

The specific examples of the present disclosure will be further described below.

The present disclosure provides an industrialized production method of a functional candy with a special effect on gastrointestinal regulation, including the following steps: step 1, preparing a yacon polysaccharide-peptide-zinc chelate with a high activity; step 2, mixing the yacon polysaccharide-peptide-zinc chelate obtained in step 1 with a chicory extract powder, a Hericium erinaceus extract powder, xylooligosaccharide, fructooligosaccharide, and water to obtain a main agent; and step 3, adding an auxiliary agent including other ingredients to the main agent to prepare the functional candy.

Step 1 specifically includes the following steps: step 1.1, cultivating the yacon, applying a zinc-rich nutrient solution to each yacon seedling during a growth process of the yacon seedlings until one week before the yacon matures; step 1.2, preparing a homogenate from a mature yacon fruit as a raw material, weighing, adding water, heating and stirring under heat preservation, adding an enzyme to conduct a reaction, filtering an obtained enzymatic hydrolysate with a centrifuge to remove residues, and conducting inactivation on the enzymatic hydrolysate; step 1.3, adding bioethanol to an inactivated enzymatic hydrolysate, allowing to stand, and filtering to obtain a yacon polysaccharide-peptide-zinc chelate crude extract; and step 1.4, redissolving the yacon polysaccharide-peptide-zinc chelate crude extract in water, and conducting an ultrafiltration membrane treatment to obtain the yacon polysaccharide-peptide-zinc chelate with a high activity.

In step 1.1, during cultivating the yacon, when the yacon seedlings grow to 10 cm to 15 cm, 50 mL to 200 mL of the zinc-rich nutrient solution is applied to each yacon seedling every 3 d to 5 d until one week before the yacon matures; the zinc-rich nutrient solution includes the following components in parts by weight: 1 part to 5 parts of a zinc fertilizer, 1 part to 5 parts of an amino acid, 20 parts to 50 parts of a compound fertilizer, 10 parts to 30 parts of sawdust, 20 parts to 30 parts of wheat bran, and 1000 parts of water; a zinc element form of the zinc fertilizer in the zinc-rich nutrient solution includes any one or more selected from the group consisting of zinc sulfate heptahydrate, a zinc-amino acid chelate, a zinc-rich yeast, and a zinc-EDTA chelate; and the zinc-rich nutrient solution is applied to the yacon seedlings by foliage spray or root fertilization.

Preferably, the yacon is Yunnan mountain yacon. Yacon seedlings are 10 cm to 12 cm high.

In step 1.2, the homogenate is prepared from the mature yacon fruit as the raw material, weighed, water is added according to a mass ratio of the raw material (w) and water (w) at 1:10 to 1:30, heated to 90° C. to 95° C. and stirred under heat preservation for 2 h to 3 h, the enzyme is added according to 0.1% to 1% of a mass of the raw material to conduct a reaction at 40° C. to 65° C. and a pH value of 4.5 to 7.0 for 80 min to 100 min, the obtained enzymatic hydrolysate is filtered with a centrifuge at 2,000 r/min to 4,000 r/min to remove the residues, and inactivation is conducted on the enzymatic hydrolysate at 90° C. to 100° C.; and the enzyme is any one or an enzyme complex of more selected from the group consisting of cellulase, hemicellulase, and pectinase.

Preferably, the yacon fruit (w) and the water (w) have a mass-to-volume ratio of 1:15 to 1:20. The enzyme is an enzyme complex of the cellulase and the pectinase at a molar ratio of 1:1 to 1:2. The inactivation is conducted at 95° C. to 100° C.

In step 1.3, 65% to 90% of the bioethanol by mass concentration is added to the inactivated enzymatic hydrolysate according to a volume-to-volume (v/v) ratio of ethanol and the enzymatic hydrolysate at 2:1 to 4:1, allowed to stand, and filtered to obtain the yacon polysaccharide-peptide-zinc chelate crude extract.

Preferably, the ethanol and the enzymatic hydrolysate have a volume-to-volume (v/v) ratio of 3:1 to 4:1.

In step 1.4, the yacon polysaccharide-peptide-zinc chelate crude extract is redissolved in water according to a mass ratio (w/w) of the yacon polysaccharide-peptide-zinc chelate crude extract and water at 1:10 to 1:30, and the ultrafiltration membrane treatment is conducted at 0.22 MPa to 0.30 MPa and 25° C. to 30° C. in a material-liquid volume flow rate of 0.08 L/min to 0.12 L/min using ultrafiltration membranes with molecular weight cut-offs of 40,000 and 30,000 separately to obtain the yacon polysaccharide-peptide-zinc chelate with a high activity and having a molecular weight of 30,000 to 40,000.

Preferably, the ultrafiltration membrane treatment is conducted at 0.25 MPa to 0.30 MPa and 28° C. to 30° C. in a material-liquid volume flow rate of 0.08 L/min to 0.10 L/min.

The present disclosure provides a preparation method of a yacon polysaccharide-peptide-zinc chelate with a high activity, and further provides a preparation method of a functional candy with a special effect on gastrointestinal regulation by the yacon polysaccharide-peptide-zinc chelate and various plant active materials.

In step 2, in parts by weight, 1 part to 5 parts of the yacon polysaccharide-peptide-zinc chelate is mixed with 10 parts to 20 parts of the chicory extract powder, 10 parts to 20 parts of the Hericium erinaceus extract powder, 5 parts to 15 parts of the xylooligosaccharide, 5 parts to 15 parts of the fructooligosaccharide, and 10 parts to 20 parts of the water, and stirred at 50° C. to 60° C. for 20 min to 30 min to obtain the main agent.

In step 3, the main agent and the auxiliary agent are processed to prepare the functional candy with a special effect on gastrointestinal regulation. A type of the candy includes any one or more selected from the group consisting of a gelatinous candy, a hard candy, and a tablet candy.

When the candy is the gelatinous candy: a gellant is stirred with water, and cooled to obtain a gelatinous candy auxiliary agent, the main agent and the auxiliary agent are mixed, stirred evenly, and an obtained material liquid is poured into a silica gel mold for molding, demolding is conducted after cooling, and the gelatinous candy is sealed and packaged under vacuum conditions; and the gellant is any one or more selected from the group consisting of carrageenan, gelatin, agar, konjac gum, pectin, and gum arabic.

Preferably, when the candy is the gelatinous candy: the gellant is stirred with an appropriate amount of water at 90° C. to 100° C. for 20 min to 30 min, and cooled to 50° C. to 60° C. to obtain the gelatinous candy auxiliary agent; the main agent and the gelatinous candy auxiliary agent as a gel liquid are mixed, stirred evenly at 95° C. to 100° C. for 15 min to 25 min, and cooled to 50° C. to 70° C., and the obtained material liquid is poured into the silica gel mold for molding, demolding is conducted after cooling for 20 min to 30 min, and the gelatinous candy is sealed and packaged under vacuum conditions.

When the candy is the hard candy: an auxiliary agent including a sugar or a sugar alcohol and the main agent are mixed uniformly by heating, and boiled at a high temperature to obtain a material liquid, and the material liquid is poured into the silica gel mold for molding, dried, and demolded to obtain the hard candy; and the sugar or the sugar alcohol is any one or more selected from the group consisting of white granulated sugar, fructose, and xylitol.

Preferably, when the candy is the hard candy: 10 parts to 15 parts of the sugar or the sugar alcohol and 1 part of the main agent are mixed uniformly with water by heating at 60° C. to 80° C., and boiled at 120° C. to 140° C. to obtain the material liquid, and the material liquid is poured into the silica gel mold for molding, dried to a moisture content of less than 6% of a mass of the candy, and demolded to obtain the hard candy.

When the candy is the tablet candy: the main agent and the auxiliary agent including the sugar or the sugar alcohol are mixed, granulated, dried, and sieved, added with the 1% lubricant by mass percentage, mixed evenly and tableted in the tablet press to obtain the tablet candy; and the sugar or the sugar alcohol is any one or more selected from the group consisting of glucose, isomaltooligosaccharide, maltitol, and erythritol.

Preferably, when the candy is the tablet candy: 3 parts to 4 parts of the main agent and 5 parts to 10 parts of the sugar or the sugar alcohol are mixed for 20 min to 30 min by stirring, granulated in a rotary automatic granulator, dried with hot air at 70° C. to 80° C., and sieved by a 60 mesh to 80 mesh sieve, added with the 1% lubricant by mass percentage, mixed evenly and tableted in the tablet press to obtain the tablet candy.

Among the auxiliary agents, such as the lubricant, specific components used and proportions can be appropriately selected by those skilled in the art.

The present disclosure further provides a functional candy with a special effect on gastrointestinal regulation produced by the method, including a functional main agent prepared by the following components in parts by weight: 1 part to 5 parts of the yacon polysaccharide-peptide-zinc chelate, 10 parts to 20 parts of the chicory extract powder, 10 parts to 20 parts of the Hericium erinaceus extract powder, 5 parts to 15 parts of the xylooligosaccharide, 5 parts to 15 parts of the fructooligosaccharide, and 10 parts to 20 parts of water; where the yacon polysaccharide-peptide-zinc chelate is a compound with a molecular weight of 30,000 to 40,000, and has 68.9% to 73.8% of a polysaccharide, 25.7% to 26.1% of a peptide, and 150 mg/100 g to 200 mg/100 g of zinc by mass percentage.

Among them, the yacon is a Compositae herb, with its roots containing a large amount of active sugars, including fructooligosaccharide-based prebiotics such as 1-kestose, nystose, and 1F-fructofuranosytnystose. The human gut contains a large amount of probiotics, and a quantity thereof can be used as a standard to measure human health; the yacon is rich in a large amount of fructooligosaccharide-based prebiotics, which can promote the natural reproduction of beneficial bacteria in the intestines to protect the health of intestines.

The functional candy with a special effect on gastrointestinal regulation and the industrialized production method thereof provided by the present disclosure will be further described below in conjunction with the examples.

### EXAMPLE 1

Step 1: during cultivating yacon, when yacon seedlings grew to 14 cm, a zinc-rich nutrient solution was applied to each yacon seedling on a leaf surface every 5 d until one week before the yacon matured; the zinc-rich nutrient solution included the following components in parts by weight: 3 parts of a zinc-rich yeast, 3 parts of an amino acid, 25 parts of a compound fertilizer, 10 parts of sawdust, 20 parts of wheat bran, and 1000 parts of water.

Step 2: a homogenate was prepared from a mature yacon fruit as a raw material, weighed, water was added according to a mass-to-volume ratio of the raw material (w) and water (w) at 1:15, heated to 90° C. and stirred under heat preservation for 2 h, an enzyme complex including cellulase and pectinase in a same molar ratio was added according to 0.1% of a mass of the raw material to conduct a reaction at 55° C. and a pH value of 4.8 for 80 min, an obtained enzymatic hydrolysate was filtered with a centrifuge at 2,000 r/min to remove residues, and inactivation was conducted on the enzymatic hydrolysate at 95° C.; where a polysaccharides extraction rate was 69.8%.

Step 3: 65% of bioethanol by concentration was added to an inactivated enzymatic hydrolysate according to according to a volume-to-volume (v/v) ratio of an ethanol and the enzymatic hydrolysate at 2:1, allowed to stand, and filtered to obtain a yacon polysaccharide-peptide-zinc chelate crude extract.

Step 4: the yacon polysaccharide-peptide-zinc chelate crude extract was redissolved in water according to a mass ratio of the yacon polysaccharide-peptide-zinc chelate crude extract (w) and water (w) at 1:15, and an ultrafiltration membrane treatment was conducted at 0.25 MPa and 28° C. in a material-liquid volume flow rate of 0.08 L/min using ultrafiltration membranes with molecular weight cut-offs of 40,000 and 30,000 separately to obtain a yacon polysaccharide-peptide-zinc chelate with a high activity and having a molecular weight of 30,000 to 40,000. After measurement, a zinc content was 164 mg/100 g, and a peptide content was 29.5%.

Step 5: in parts by weight, 1 part of the yacon polysaccharide-peptide-zinc chelate, 10 parts of a chicory extract powder, 10 parts of a Hericium erinaceus extract powder, 15 parts of xylooligosaccharide, 15 parts of fructooligosaccharide, and 20 parts of water were mixed by stirring at 50° C. for 30 min to prepare a main agent.

Step 6: 2 parts of pectin was poured into an appropriate amount of water, heated in a water bath, and slowly added with 1 part of gum arabic while stirring continuously, until the gel was completely dissolved with no particles, and incubation was conducted at 55° C. for 20 min to obtain a gel solution.

Step 7: the main agent in step 5 and the gel solution in step 6 were stirred uniformly at 95° C. for 20 min, and cooled to 55° C. An obtained material liquid was poured into a silica gel mold for molding, demolding was conducted after cooling for 30 min, and a product was sealed and packaged under vacuum conditions.

### EXAMPLE 2

Step 1: during cultivating yacon, when yacon seedlings grew to 12 cm, a zinc-rich nutrient solution was applied to each yacon seedling on a root every 4 d until one week before the yacon matured; the zinc-rich nutrient solution included the following components in parts by weight: 3 parts of a zinc-EDTA chelate, 5 parts of an amino acid, 30 parts of a compound fertilizer, 10 parts of sawdust, 25 parts of wheat bran, and 1000 parts of water.

Step 2: a homogenate was prepared from a mature yacon fruit as a raw material, weighed, water was added according to a mass-to-volume ratio of the raw material (w) and water (w) at 1:20, heated to 92° C. and stirred under heat preservation for 2.5 h, an enzyme complex including cellulase and pectinase in a molar ratio of 1:2 was added according to 0.3% of a mass of the raw material to conduct a reaction at 55° C. and a pH value of 5.0 for 80 min, an obtained enzymatic hydrolysate was filtered with a centrifuge at 3,000 r/min to remove residues, and inactivation was conducted on the enzymatic hydrolysate at 98° C.; where a polysaccharides extraction rate was 70.1%.

Step 3: 80% of bioethanol by concentration was added to an inactivated enzymatic hydrolysate according to a volume-to-volume (v/v) ratio of an ethanol and the enzymatic hydrolysate at 3:1, allowed to stand, and filtered to obtain a yacon polysaccharide-peptide-zinc chelate crude extract.

Step 4: the yacon polysaccharide-peptide-zinc chelate crude extract was redissolved in water according to a mass ratio of the yacon polysaccharide-peptide-zinc chelate crude extract (w) and water (w) at 1:20, and an ultrafiltration membrane treatment was conducted at 0.30 MPa and 30° C. in a material-liquid volume flow rate of 0.10 L/min using ultrafiltration membranes with molecular weight cut-offs of 40,000 and 30,000 separately to obtain a yacon polysaccharide-peptide-zinc chelate with a high activity and having a molecular weight of 30,000 to 40,000. After measurement, a zinc content was 180mg/100 g, and a peptide content was 29.6%.

Step 5: in parts by weight, 3 parts of the yacon polysaccharide-peptide-zinc chelate, 20 parts of a chicory extract powder, 20 parts of a Hericium erinaceus extract powder, 15 parts of xylooligosaccharide, 15 parts of fructooligosaccharide, and 20 parts of water were mixed by stirring at 50° C. for 30 min to prepare a main agent.

Step 6: 2 parts of carrageenan was poured into an appropriate amount of water, heated in a water bath, and slowly added with 1 part of gelatin while stirring continuously, until the gel was completely dissolved with no particles, and incubation was conducted at 55° C. for 10min to obtain a gel solution.

Step 7: the main agent and the gel solution were stirred uniformly at 95° C. for 20 min, and cooled to 55° C. An obtained material liquid was poured into a silica gel mold for molding, demolding was conducted after cooling for 30 min, and a product was sealed and packaged under vacuum conditions.

### EXAMPLE 3

Step 1: during cultivating yacon, when yacon seedlings grew to 15 cm, a zinc-rich nutrient solution was applied to each yacon seedling on a root every 3 d until one week before the yacon matured; the zinc-rich nutrient solution included the following components in parts by weight: 5 parts of zinc sulfate heptahydrate, 5 parts of an amino acid, 30 parts of a compound fertilizer, 10 parts of sawdust, 20 parts of wheat bran, and 1000 parts of water.

Step 2: a homogenate was prepared from a mature yacon fruit as a raw material, weighed, water was added according to a mass-to-volume ratio of the raw material (w) and water (w) at 1:20, heated to 95° C. and stirred under heat preservation for 3 h, an enzyme complex including cellulase and pectinase in a molar ratio of 1:2 was added according to 0.05% of a mass of the raw material to conduct a reaction at 55° C. and a pH value of 6.0 for 80 min, an obtained enzymatic hydrolysate was filtered with a centrifuge at 2,000 r/min to remove residues, and inactivation was conducted on the enzymatic hydrolysate at 98° C.; where a polysaccharides extraction rate was 72.5%.

Step 3: 80% of bioethanol by concentration was added to an inactivated enzymatic hydrolysate according to a volume-to-volume (v/v) ratio of an ethanol and the enzymatic hydrolysate at 3:1, allowed to stand, and filtered to obtain a yacon polysaccharide-peptide-zinc chelate crude extract.

Step 4: the yacon polysaccharide-peptide-zinc chelate crude extract was redissolved in water according to a mass ratio of water polysaccharide-peptide-zinc chelate crude extract (w) and water (w) at 1:30, and an ultrafiltration membrane treatment was conducted at 0.30 MPa and 30° C. in a material-liquid volume flow rate of 0.10 L/min using ultrafiltration membranes with molecular weight cut-offs of 40,000 and 30,000 separately to obtain a yacon polysaccharide-peptide-zinc chelate with a high activity and having a molecular weight of 30,000 to 40,000. After measurement, a zinc content was 188mg/100 g, and a peptide content was 29.5%.

Step 5: in parts by weight, 4 parts of the yacon polysaccharide-peptide-zinc chelate, 12 parts of a chicory extract powder, 20 parts of a Hericium erinaceus extract powder, 15 parts of xylooligosaccharide, 15 parts of fructooligosaccharide, and 20 parts of water were mixed by stirring at 50° C. for 30 min to prepare a main agent.

Step 6: 2 parts of pectin was poured into an appropriate amount of water, heated in a water bath, and slowly added with 1 part of gelatin while stirring continuously, until the gel was completely dissolved with no particles, and incubation was conducted at 55° C. for 10 min to obtain a gel solution.

Step 7: the main agent and the gel solution were stirred uniformly at 95° C. for 20 min, and cooled to 55° C. An obtained material liquid was poured into a silica gel mold for molding, demolding was conducted after cooling for 30 min, and a product was sealed and packaged under vacuum conditions.

### EXAMPLE 4

Step 1: the yacon polysaccharide-peptide-zinc chelate in Example 2 was obtained, including the following components in parts by weight: 3 parts of the yacon polysaccharide-peptide-zinc chelate, 10 parts of a chicory extract powder, 10 parts of a Hericium erinaceus extract powder, 15 parts of xylooligosaccharide, 15 parts of fructooligosaccharide, and 20 parts of water were mixed by stirring at 55° C. for 30 min to prepare a main agent.

Step 2: in parts by weight, 15 parts of white granulated sugar, 1 part of fructose, and 1 part of the main agent were mixed uniformly with an appropriate amount of water by heating at 80° C., and boiled at 120° C. to obtain a material liquid, and the material liquid was poured into a silica gel mold for molding, dried to a moisture content of 5%, and demolded to obtain a hard candy.

### EXAMPLE 5

Step 1: the yacon polysaccharide-peptide-zinc chelate in Example 3 was obtained, including the following components in parts by weight: 3 parts of the yacon polysaccharide-peptide-zinc chelate, 12 parts of a chicory extract powder, 20 parts of a Hericium erinaceus extract powder, 15 parts of xylooligosaccharide, 15 parts of fructooligosaccharide, and 20 parts of water were mixed by stirring at 50° C. for 30 min to prepare a main agent.

Step 2: in parts by weight, 5 parts of maltitol, 3 parts of glucose, and 3 parts of the main agent were mixed for 30 min by stirring, granulated in a rotary automatic granulator, dried with hot air at 80° C., and sieved by a 60 mesh sieve, added with a 1% lubricant, mixed evenly and tableted in the tablet press to obtain the tablet candy.

### Experimental Results+−

### (1) Mice Laxative Function Experiment

50 Kunming mice, half male and half male, 8-week-old, were adaptively reared for one week, weighing 18 g to 22 g, and then randomly divided into five groups with 10 mice in each group, each of which was set up as a blank control group, a model group, a low-dosage group, a middle-dosage group, and a high-dosage group, a model group, and an administration group; the mice were fed with rice and deprived of water to establish a constipation model. Except for the blank and model groups, the functional candy main agent of Example 3 was administered by gavage every morning, where dosages of the low-dosage group, middle-dosage group and high-dosage group were 2 mg/(kg·BW·d), 4 mg/(kg·BW·d) and 6 mg/(kg·BW·d), respectively; meanwhile, the blank and model groups were given the same weight of a normal saline; the feeding were conducted for 5 d with free food and drink. A small intestine exercise experiment and a defecation experiment were designed, and water contents of feces and intestines were measured, and a laxative effect of zinc-rich yacon candy on constipated mice was determined. The test results were shown in Table 1 and Table 2.

A calculation method of an ink propulsion length was as follows:

$$Ink propulsion rate ( \% ) = \frac{Ink propulsion length ( cm )}{Small intestine total length ( cm )} \times 100$$

TABLE 1
Effects of different dosages of zinc-rich yacon candy on intestinal propulsion rate in mice

| | | | Small | | |
|---|---|---|---|---|---|
| | Number of | Dosage/ | intestine total | Propulsion | Small intestinal |
| Group | Animals | (mg/(kg*BW*d)) | length/cm | distance/cm | propulsion rate/% |
| Control group | 10 | 0 | 53.4 ± 2.5 | 41.4 ± 5.6 | 77.5 ± 10.6 |
| Model control group | 10 | 0 | 54.2 ± 2.8 | 25.3 ± 5.4 | 46.7 ± 9.5 |
| Low dose group | 10 | 2 | 54.6 ± 3.1 | 28.6 ± 4.5 | 52.4 ± 8.9 |
| Middle dose group | 10 | 4 | 54.8 ± 2.4 | 32.5 ± 5.1 | 59.3 ± 9.4 |
| High dose group | 10 | 6 | 54.1 ± 2.4 | 36.7 ± 4.8 | 67.9 ± 10.3 |

TABLE 2
Effects of different dosages of zinc-rich yacon candy on defecation in mice

| | | | First | | |
|---|---|---|---|---|---|
| | Number of | Dosage/ | defecation | 6 h fecal | 6 h fecal |
| Group | Animals | (mg/(kg*BW*d)) | time/min | pellets/grain | pellets/mg |
| Control group | 10 | 0 | 106.8 ± 18.5 | 26.3 ± 2.8 | 425 ± 85 |
| Model control group | 10 | 0 | 186.6 ± 12.5 | 20.3 ± 4.3 | 334 ± 76 |
| Low dose group | 10 | 2 | 158.4 ± 15.6 | 21.6 ± 3.8 | 406 ± 82 |
| Middle dose group | 10 | 4 | 143.3 ± 16.5 | 22.4 ± 3.1 | 375 ± 69 |
| High dose group | 10 | 6 | 126.7 ± 14.6 | 25.8 ± 2.7 | 347 ± 84 |

The experimental results showed that the small intestine ink propulsion rate in each dose group increased significantly, and the effect of medium and high dosages was the most obvious; the main agent of zinc-rich yacon candy had a promoting effect on the small intestine. The constipation model mice were intervened with different dosages of the main agent, and the results showed that the time to first defecation time, the number of feces in 6 h and the weight of feces in 6 h were significantly changed. Therefore, the main agent of zinc-rich yacon candy can effectively promote the intestinal peristalsis of mice, and has a significant effect on promoting defecation, especially at medium and high dosages.

### (2) Crowd Laxative Experiment

The zinc-rich functional candy with laxatives obtained in Example 1 to 3 was used for 100 people with constipation to observe. Among them, women accounted for 60%, men accounted for 40%; 25 to 35 years old accounted for 30%; 35 to 45 years old accounted for 50%; 45 to 55 years old accounted for 10%.

The results show that the effective rate of the functional candy with laxatives obtained by the present disclosure to constipation patients was more than 85%; among them, 90% of the people had an increase in the number of defecation, 85% of the people had a smoother defecation, 87% of the people had an increase in the number of defecation, and 85% of the people had the feeling of bloating disappear.

The followings were clinical effects of some typical cases of the yacon candy with laxative effect obtained by the present disclosure.

Case 1:

Ms. Wang, 46 years old, had constipation for 6 years, and defecation was only once every 2 d to 3 d; after taking the yacon candy with laxative effect obtained in Example 3, defecation was increased once within 8 h on the same day; after taking for four weeks, the constipation was ameliorated, at once a day.

Case 2:

Mr. Ouyang, 38 years old, had constipation for 3 years, and defecation was only once every 3 d; after taking the yacon candy with laxative effect obtained in Example 3, defecation was increased once within 10 h on the same day; after taking for three weeks, the constipation was ameliorated, at once a day.

Case 3:

Ms. Zhang, 49 years old, had constipation for 8 years, and defecation was only once every 4 d; after taking the yacon candy with laxative effect obtained in Example 2, defecation was increased once within 12 h on the same day; after taking for four weeks, the constipation was ameliorated, at once every 2 days.

Case 4:

Mr. Qin, 55 years old, had constipation for 5 years, and defecation was only once every 3 d; after taking the yacon candy with laxative effect obtained in Example 2, defecation was increased once within 7 h on the same day; after taking for three weeks, the constipation was ameliorated, at once every 2 days.

Case 5:

Ms. Li, 32 years old, had constipation for 2 years, and defecation was only once every 2 d; after taking the yacon candy with laxative effect obtained in Example 1, defecation was increased once within 5 h on the same day; after taking for two weeks, the constipation was ameliorated, at once a day.

Case 6:

Mr. Zheng, 25 years old, had constipation for 3 years, and defecation was only once every 2 d; after taking the yacon candy with laxative effect obtained in Example 1, defecation was increased once within 4 h on the same day; after taking for two weeks, the constipation was ameliorated, at once a day.

Case 7:

Ms. He, 33 years old, had constipation for 1 year, and defecation was only once every 2 d; after taking the yacon functional candy with laxative effect obtained in Example 4, defecation was increased once within 12 h on the same day; after taking for two weeks, the constipation was ameliorated, at once every 1.5 days.

Above result shows that, after taking zinc-rich yacon candy, constipation patient obviously increases stool frequency and obviously shortens defecation time, with a significant curative effect; after long-term constipation patient continuously takes for four weeks, constipation symptom basically disappears.

In the present disclosure, in the functional candy with a special effect on gastrointestinal regulation and an industrialized production method thereof, the zinc element necessary for the human body is converted into a functional chelate compound with a higher biological utility, supplemented by various natural plant activities. The ingredients are carefully formulated to prepare the functional candy that regulates the action of the gastrointestinal tract, and has a significant effect on protecting the health of the gastrointestinal tract.

In the present disclosure, the functional candy with a special effect on gastrointestinal regulation has an effective rate of more than 85% in constipated people, including increased defecation volume, increased defecation frequency, and smoother defecation sensation; the candy has green and natural raw materials, no side effects, and suitability for long-term use, as well as a moderately sweet and sour taste, which is easy to carry; the preparation method thereof is mature, easy to operate and implement, and suitable for industrial production.

In the present disclosure, the candy is rich in yacon polysaccharide-peptide-zinc chelate with a molecular weight of 30,000 to 40,000, and has 68.9% to 73.8% of a polysaccharide, 25.7% to 26.1% of a peptide, and 150 mg/100 g to 200 mg/100 g of zinc by mass percentage. By applying the zinc-rich nutrient solution to the yacon seedlings, the yacon fruit rich in biological zinc is obtained; mature yacon fruits are selected as a raw material, and cellulase, pectinase or hemicellulase acts on yacon fruit cell wall, promoting effective ingredient precipitation, which significantly improves active ingredients in the extract, especially the content of polysaccharide; and separation is conducted with a two-stage ultrafiltration membrane to obtain the yacon polysaccharide-peptide-zinc chelate with a molecular weight of 30,000-40,000. In parts by weight, 1 part to 5 parts of the yacon polysaccharide-peptide-zinc chelate is mixed with 10 parts to 20 parts of the chicory extract powder, 10 parts to 20 parts of the Hericium erinaceus extract powder, 5 parts to 15 parts of the xylooligosaccharide, 5 parts to 15 parts of the fructooligosaccharide, and 10 parts to 20 parts of the water, to obtain the main agent, and the main agent and the candy accessories are mixed and boiled to prepare the functional candy with a special effect on gastrointestinal regulation. The yacon polysaccharide-peptide-zinc chelate-rich functional candy has desirable taste, moderate sweetness and sourness, and can significantly enhance intestinal peristalsis and regulate the vitality of intestinal flora; animal and human experiments have shown that the candy can effectively relieve and treat constipation after taking, with a remarkable effect. The highly-active yacon polysaccharide-peptide-zinc chelate can be applied to the field of health care products.

Although the content of the present disclosure has been described in detail through the aforementioned preferred embodiments, it should be recognized that the above description should not be considered as limiting the present disclosure. Upon reading the aforementioned content, it will be apparent to those skilled in the art that various modifications and alternations to the present disclosure can be made. Therefore, the claimed scope of the present disclosure shall be defined by the appended claims.

1. An industrialized production method of a functional candy with a special effect on gastrointestinal regulation, comprising the following steps:
step 1, preparing a yacon polysaccharide-peptide-zinc chelate with a high activity;
step 2, mixing the yacon polysaccharide-peptide-zinc chelate obtained in step 1 with a chicory extract powder, a Hericium erinaceus extract powder, xylooligosaccharide, fructooligosaccharide, and water to obtain a main agent; and
step 3, adding an auxiliary agent comprising other ingredients to the main agent to prepare the functional candy.

2. The industrialized production method of a functional candy with a special effect on gastrointestinal regulation according to claim 1, wherein step 1 specifically comprises the following steps:
step 1.1, cultivating the yacon, applying a zinc-rich nutrient solution to each yacon seedling during a growth process of the yacon seedlings until one week before the yacon matures;
step 1.2, preparing a homogenate from a mature yacon fruit as a raw material, weighing, adding water, heating and stirring under heat preservation, adding an enzyme to conduct a reaction, filtering an obtained enzymatic hydrolysate with a centrifuge to remove residues, and conducting inactivation on the enzymatic hydrolysate;
step 1.3, adding bioethanol to an inactivated enzymatic hydrolysate, allowing to stand, and filtering to obtain a yacon polysaccharide-peptide-zinc chelate crude extract; and
step 1.4, redissolving the yacon polysaccharide-peptide-zinc chelate crude extract in water, and conducting an ultrafiltration membrane treatment to obtain the yacon polysaccharide-peptide-zinc chelate with a high activity.

3. The industrialized production method of a functional candy with a special effect on gastrointestinal regulation according to claim 2, wherein in step 1.1, during cultivating the yacon, when the yacon seedlings grow to 10 cm to 15 cm, 50 mL to 200 mL of the zinc-rich nutrient solution is applied to each yacon seedling every 3 d to 5 d until one week before the yacon matures; the zinc-rich nutrient solution comprises the following components in parts by weight: 1 part to 5 parts of a zinc fertilizer, 1 part to 5 parts of an amino acid, 20 parts to 50 parts of a compound fertilizer, 10 parts to 30 parts of sawdust, 20 parts to 30 parts of wheat bran, and 1000 parts of water; a zinc element form of the zinc fertilizer in the zinc-rich nutrient solution comprises any one or more selected from the group consisting of zinc sulfate heptahydrate, a zinc-amino acid chelate, a zinc-rich yeast, and a zinc-EDTA chelate; and the zinc-rich nutrient solution is applied to the yacon seedlings by foliage spray or root fertilization.

4. The industrialized production method of a functional candy with a special effect on gastrointestinal regulation according to claim 2, wherein in step 1.2, the homogenate is prepared from the mature yacon fruit as the raw material, weighed, water is added according to a mass-to-volume ratio of the raw material and water at 1:10 to 1:30, heated to 90° C. to 95° C. and stirred under heat preservation for 2 h to 3 h, the enzyme is added according to 0.1% to 1% of a mass of the raw material to conduct a reaction at 40° C. to 65° C. and a pH value of 4.5 to 7.0 for 80 min to 100 min, the obtained enzymatic hydrolysate is filtered with a centrifuge at 2,000 r/min to 4,000 r/min to remove the residues, and inactivation is conducted on the enzymatic hydrolysate at 90° C. to 100° C.; and the enzyme is any one or an enzyme complex of more selected from the group consisting of cellulase, hemicellulase, and pectinase.

5. The industrialized production method of a functional candy with a special effect on gastrointestinal regulation according to claim 2, wherein in step 1.3, 65% to 90% of the bioethanol by mass concentration is added to the inactivated enzymatic hydrolysate according to a volume ratio of ethanol and the enzymatic hydrolysate at 2:1 to 4:1, allowed to stand, and filtered to obtain the yacon polysaccharide-peptide-zinc chelate crude extract.

6. The industrialized production method of a functional candy with a special effect on gastrointestinal regulation according to claim 2, wherein in step 1.4, the yacon polysaccharide-peptide-zinc chelate crude extract is redissolved in water according to a mass ratio of the yacon polysaccharide-peptide-zinc chelate crude extract and water at 1:10 to 1:30, and the ultrafiltration membrane treatment is conducted at 0.22 MPa to 0.30 MPa and 25° C. to 30° C. in a material-liquid volume flow rate of 0.08 L/min to 0.12 L/min using ultrafiltration membranes with molecular weight cut-offs of 40,000 and 30,000 separately to obtain the yacon polysaccharide-peptide-zinc chelate with a high activity and having a molecular weight of 30,000 to 40,000.

7. The industrialized production method of a functional candy with a special effect on gastrointestinal regulation according to claim 1, wherein in step 2, in parts by weight, 1 part to 5 parts of the yacon polysaccharide-peptide-zinc chelate are mixed with 10 parts to 20 parts of the chicory extract powder, 10 parts to 20 parts of the Hericium erinaceus extract powder, 5 parts to 15 parts of the xylooligosaccharide, 5 parts to 15 parts of the fructooligosaccharide, and 10 parts to 20 parts of the water, and stirred at 50° C. to 60° C. for 20 min to 30 min to obtain the main agent.

8. The industrialized production method of a functional candy with a special effect on gastrointestinal regulation according to claim 1, wherein in step 3, a type of the candy comprises any one or more selected from the group consisting of a gelatinous candy, a hard candy, and a tablet candy.

9. The industrialized production method of a functional candy with a special effect on gastrointestinal regulation according to claim 8, wherein when the candy is the gelatinous candy: a gellant is stirred with water, and cooled to obtain a gelatinous candy auxiliary agent, the main agent and the auxiliary agent are mixed, stirred evenly, and an obtained material liquid is poured into a silica gel mold for molding, demolding is conducted after cooling, and the gelatinous candy is sealed and packaged under vacuum conditions; and the gellant is any one or more selected from the group consisting of carrageenan, gelatin, agar, konjac gum, pectin, and gum arabic; when the candy is the hard candy: an auxiliary agent comprising a sugar or a sugar alcohol and the main agent are mixed uniformly by heating, and boiled at a high temperature to obtain a material liquid, and the material liquid is poured into the silica gel mold for molding, dried, and demolded to obtain the hard candy; and the sugar or the sugar alcohol is any one or more selected from the group consisting of white granulated sugar, fructose, and xylitol; when the candy is the tablet candy: the main agent and the auxiliary agent comprising the sugar or the sugar alcohol are mixed, granulated, dried, and sieved, added with a 1% lubricant by mass percentage, mixed evenly and tableted in a tablet press to obtain the tablet candy; and the sugar or the sugar alcohol is any one or more selected from the group consisting of glucose, isomaltooligosaccharide, maltitol, and erythritol.

10. A functional candy with a special effect on gastrointestinal regulation produced by the method according to claim 1, comprising a functional main agent prepared by the following components in parts by weight: 1 part to 5 parts of the yacon polysaccharide-peptide-zinc chelate, 10 parts to 20 parts of the chicory extract powder, 10 parts to 20 parts of the Hericium erinaceus extract powder, 5 parts to 15 parts of the xylooligosaccharide, 5 parts to 15 parts of the fructooligosaccharide, and 10 parts to 20 parts of water; wherein the yacon polysaccharide-peptide-zinc chelate is a compound with a molecular weight of 30,000 to 40,000, and has 68.9% to 73.8% of a polysaccharide, 25.7% to 26.1% of a peptide, and 150 mg/100 g to 200 mg/100 g of zinc by mass percentage.

11. The functional candy with a special effect on gastrointestinal regulation according to claim 10, wherein step 1 specifically comprises the following steps:
step 1.1, cultivating the yacon, applying a zinc-rich nutrient solution to each yacon seedling during a growth process of the yacon seedlings until one week before the yacon matures;
step 1.2, preparing a homogenate from a mature yacon fruit as a raw material, weighing, adding water, heating and stirring under heat preservation, adding an enzyme to conduct a reaction, filtering an obtained enzymatic hydrolysate with a centrifuge to remove residues, and conducting inactivation on the enzymatic hydrolysate;
step 1.3, adding bioethanol to an inactivated enzymatic hydrolysate, allowing to stand, and filtering to obtain a yacon polysaccharide-peptide-zinc chelate crude extract; and
step 1.4, redissolving the yacon polysaccharide-peptide-zinc chelate crude extract in water, and conducting an ultrafiltration membrane treatment to obtain the yacon polysaccharide-peptide-zinc chelate with a high activity.

12. The functional candy with a special effect on gastrointestinal regulation according to claim 11, wherein in step 1.1, during cultivating the yacon, when the yacon seedlings grow to 10 cm to 15 cm, 50 mL to 200 mL of the zinc-rich nutrient solution is applied to each yacon seedling every 3 d to 5 d until one week before the yacon matures; the zinc-rich nutrient solution comprises the following components in parts by weight: 1 part to 5 parts of a zinc fertilizer, 1 part to 5 parts of an amino acid, 20 parts to 50 parts of a compound fertilizer, 10 parts to 30 parts of sawdust, 20 parts to 30 parts of wheat bran, and 1000 parts of water; a zinc element form of the zinc fertilizer in the zinc-rich nutrient solution comprises any one or more selected from the group consisting of zinc sulfate heptahydrate, a zinc-amino acid chelate, a zinc-rich yeast, and a zinc-EDTA chelate; and the zinc-rich nutrient solution is applied to the yacon seedlings by foliage spray or root fertilization.

13. The functional candy with a special effect on gastrointestinal regulation according to claim 11, wherein in step 1.2, the homogenate is prepared from the mature yacon fruit as the raw material, weighed, water is added according to a mass-to-volume ratio of the raw material and water at 1:10 to 1:30, heated to 90° C. to 95° C. and stirred under heat preservation for 2 h to 3 h, the enzyme is added according to 0.1% to 1% of a mass of the raw material to conduct a reaction at 40° C. to 65° C. and a pH value of 4.5 to 7.0 for 80 min to 100 min, the obtained enzymatic hydrolysate is filtered with a centrifuge at 2,000 r/min to 4,000 r/min to remove the residues, and inactivation is conducted on the enzymatic hydrolysate at 90° C. to 100° C.; and the enzyme is any one or an enzyme complex of more selected from the group consisting of cellulase, hemicellulase, and pectinase.

14. The functional candy with a special effect on gastrointestinal regulation according to claim 11, wherein in step 1.3, 65% to 90% of the bioethanol by mass concentration is added to the inactivated enzymatic hydrolysate according to a volume ratio of ethanol and the enzymatic hydrolysate at 2:1 to 4:1, allowed to stand, and filtered to obtain the yacon polysaccharide-peptide-zinc chelate crude extract.

15. The functional candy with a special effect on gastrointestinal regulation according to claim 11, wherein in step 1.4, the yacon polysaccharide-peptide-zinc chelate crude extract is redissolved in water according to a mass ratio of the yacon polysaccharide-peptide-zinc chelate crude extract and water at 1:10 to 1:30, and the ultrafiltration membrane treatment is conducted at 0.22 MPa to 0.30 MPa and 25° C. to 30° C. in a material-liquid volume flow rate of 0.08 L/min to 0.12 L/min using ultrafiltration membranes with molecular weight cut-offs of 40,000 and 30,000 separately to obtain the yacon polysaccharide-peptide-zinc chelate with a high activity and having a molecular weight of 30,000 to 40,000.

16. The functional candy with a special effect on gastrointestinal regulation according to claim 10, wherein in step 2, in parts by weight, 1 part to 5 parts of the yacon polysaccharide-peptide-zinc chelate are mixed with 10 parts to 20 parts of the chicory extract powder, 10 parts to 20 parts of the Hericium erinaceus extract powder, 5 parts to 15 parts of the xylooligosaccharide, 5 parts to 15 parts of the fructooligosaccharide, and 10 parts to 20 parts of the water, and stirred at 50° C. to 60° C. for 20 min to 30 min to obtain the main agent.

17. The functional candy with a special effect on gastrointestinal regulation according to claim 10, wherein in step 3, a type of the candy comprises any one or more selected from the group consisting of a gelatinous candy, a hard candy, and a tablet candy.

18. The functional candy with a special effect on gastrointestinal regulation according to claim 17, wherein when the candy is the gelatinous candy: a gellant is stirred with water, and cooled to obtain a gelatinous candy auxiliary agent, the main agent and the auxiliary agent are mixed, stirred evenly, and an obtained material liquid is poured into a silica gel mold for molding, demolding is conducted after cooling, and the gelatinous candy is sealed and packaged under vacuum conditions; and the gellant is any one or more selected from the group consisting of carrageenan, gelatin, agar, konjac gum, pectin, and gum arabic; when the candy is the hard candy: an auxiliary agent comprising a sugar or a sugar alcohol and the main agent are mixed uniformly by heating, and boiled at a high temperature to obtain a material liquid, and the material liquid is poured into the silica gel mold for molding, dried, and demolded to obtain the hard candy; and the sugar or the sugar alcohol is any one or more selected from the group consisting of white granulated sugar, fructose, and xylitol; when the candy is the tablet candy: the main agent and the auxiliary agent comprising the sugar or the sugar alcohol are mixed, granulated, dried, and sieved, added with a 1% lubricant by mass percentage, mixed evenly and tableted in a tablet press to obtain the tablet candy; and the sugar or the sugar alcohol is any one or more selected from the group consisting of glucose, isomaltooligosaccharide, maltitol, and erythritol.
